(19) United States
(12) Patent Application Publication
(10) Pub. No.: US 2022/0105326 A1
(43) Pub. Date: Apr. 7, 2022

(54) EXTENDED RELEASE DEVICES AND THERAPEUTICS FOR LONG TERM TREATMENT OF URINARY TRACT INFECTION IN-VIVO

(71) Applicant: Dmitri Petrychenko, Staten Island, NY (US)

(72) Inventor: Dmitri Petrychenko, Staten Island, NY (US)

(21) Appl. No.: 17/493,372

(22) Filed: Oct. 4, 2021

Related U.S. Application Data
(60) Provisional application No. 63/086,738, filed on Oct. 2, 2020.

(51) Int. Cl. A61M 31/00; A61F 2/04; A61F 2/07

(52) U.S. Cl. CPC A61M 31/002; A61F 2/042; A61M 2210/1085; A61M 2205/04; A61F 2/07

(57) ABSTRACT

Devices and extended release therapeutics for the in-vivo long term treatments urinary tract infection.

### CROSS REFERENCE TO RELATED APPLICATION

The present application claims priority to U.S. Provisional Patent Application No. 63/086,738, filed on Oct. 2, 2020 the entirety which is incorporated herein by reference.

### DESCRIPTION OF RELATED ART

Chronic urinary tract infections are often hard to treat since they often respond marginally to oral antibiotics. A urinary tract infection can also spread to the blood stream, causing life-threatening septicemia. In patients that are immunologically compromised or paralyzed, due to a spinal cord injury, for example, urinary tract infections are a major problem since oral antibiotics do not function as a prophylactic to the infection. Economic benefits may also accrue attributable to a reduced need for intravenous antibiotics, hospitalization, and invasive procedures to treat urinary tract infections.

Accordingly, it would be desirable to provide a drug delivery system which avoids one or more of the drawbacks mentioned above with injection, insertion or transdermal delivery or for the treatment of bladder urinary tract infections.

### SUMMARY

The present disclosure is related to extended release therapeutic agents such as antibiotics that can be delivered via a urinary bladder implant, which are activated by inflammatory PH and inflammatory mediators. In certain embodiments, the implantable device is selected from stents, grafts, stent-grafts, catheters, shunts, closure devices, valves, and particles.

The terms “biologically degradable” (or “biodegradable”), “biologically erodable” (or “bioerodable”), “biologically absorbable” (or “bioabsorbable”), and “biologically resorbable” (or “bioresorbable”), in reference to polymers and coatings, are used interchangeably and refer to polymers and coatings that are capable of being completely or substantially completely degraded, dissolved, and/or eroded over time when exposed to physiological conditions and can be gradually resorbed, absorbed and/or eliminated by the body, or that can be degraded into fragments that can pass through the kidney membrane of an animal (e.g., a human), e.g., fragments having a molecular weight of about 40,000 Daltons (40 kDa) or less. The process of breaking down and eventual absorption and elimination of the polymer or coating can be caused by, e.g., hydrolysis, metabolic processes, oxidation, enzymatic processes, bulk or surface erosion, and the like. Conversely, a “biostable” polymer or coating refers to a polymer or coating that is not biodegradable.

“Complete degradation” of a polymer or a polymeric material (e.g., a polymeric coating) means that the polymer or the polymeric material loses at least about 95% of its mass over a period of time.

“Substantially complete degradation” of a polymer or a polymeric material (e.g., a polymeric coating) means that the polymer or the polymeric material loses at least about 75% of its mass over a period of time. In certain embodiments, “substantially complete degradation” of a polymer or a polymeric material can mean that the polymer or the polymeric material loses at least about 80% of its mass, or at least about 85% of its mass, or at least about 90% of its mass, or at least about 95% of its mass over a period of time.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses those cases where the physiological conditions (e.g., body temperature) of an animal are not considered “normal”.

### BRIEF DESCRIPTION OF THE DRAWINGS

Embodiments are illustrated in the figures of the accompanying drawings, which are meant to be exemplary and not limiting, and in which like references are intended to refer to like or corresponding things.

FIG. 1 shows an embodiment of an implant apparatus.

FIG. 2 is a cross-sectional view of the implant apparatus.

### DETAILED DESCRIPTION OF THE EMBODIMENTS

Embodiments disclosed herein provide devices and methods for a urinary bladder implant apparatus and extended release therapeutics for urinary tract infection.

In one embodiment, described is an implant apparatus comprising a container containing a therapeutic agent that has been physically trapped, or covalently or ionically immobilized, in a biodegradable matrix. The therapeutic agents can be physically trapped in the matrix by mixing, ionic bonding on the matrix and/or covalent bonding via irradiation or through the use of cross-linkers including, but not limited to, glutaraldehyde, polyethylene glycol epoxide or ethylene oxide. In an embodiment, the biodegradable matrix is in the form of a cylinder. The sides of the cylinder preferably include an impermeable coating. One or both ends of the cylinder can be open to the tissue, or the cylinder ends may be covered with a water-permeable membrane.

An element of the device is the biodegradable matrix. A biodegradable matrix, as used herein, is a matrix made of materials, usually polymers, which are degradable by enzymatic or acid/base hydrolysis. Once inserted into the body, the matrix is acted upon by enzymes and body fluids. Biodegradation, as used herein, is a process which is different from the process of dissolution. Dissolution occurs as a function of the solute properties of the material. With a dissolving matrix, the therapeutic agent is released as the matrix dissolves. Thus, the length of time the device will release the therapeutic agent is a function of the amount of therapeutic agent within the matrix. For long-term release, the device would have to be quite large to provide a continuous supply of the therapeutic agent. A matrix which is biodegradable only releases the therapeutic agent as the matrix is acted upon by body fluids or enzymes.

The biodegradability of the matrix can control the rate of release of the therapeutic agent. The therapeutic agent does not diffuse out of the matrix, but is only released as the matrix is biodegraded. In an embodiment, because the matrix is biodegradable, there is no residue of the matrix remaining when the entire dose of the therapeutic agent has been delivered. Because there is no residual matrix remaining, there is no need to remove the device.

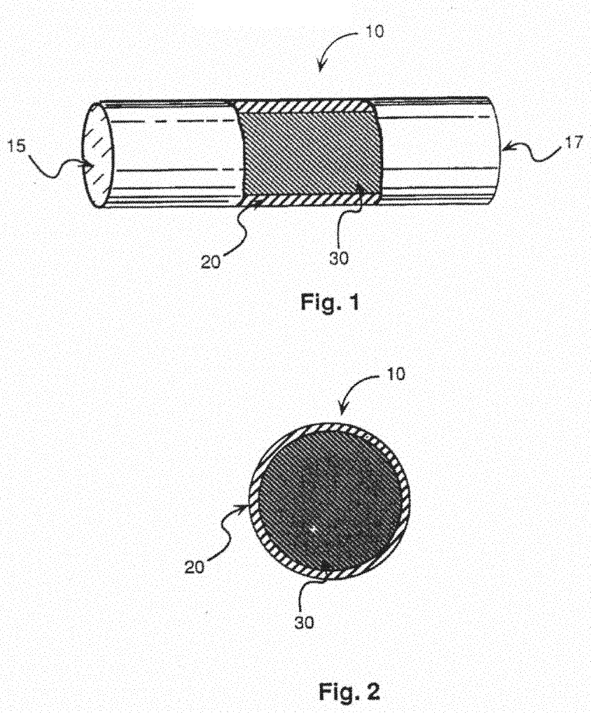

FIG. 1 shows an embodiment of an implant apparatus as shown and described in U.S. Pat. No. 5,629,008, the entirety of which is incorporated by reference herein. As shown in FIG. 1, the apparatus comprises a cylinder 10 including a wall 20 that is preferably non-permeable to macromolecules such as proteins and cells. The cylinder wall 20 can be made of medical-grade silicone, stainless steel, titanium, gold, or plastics. The cylinder 10 is filled with a biodegradable matrix 30 with a therapeutic admixed therein. The ends 15, 17 of the cylinder 10 are preferably open to the tissue. Alternatively, the ends 15, 17 of the cylinder can be covered with a membrane that is permeable to water, macromolecules and optionally to cells. In another embodiment of the present invention, one end 15 of the cylinder 10 can be sealed with a material that is impermeable to enzymes and cells and the other end of the cylinder 10 can be open to tissue. FIG. 2 is a cross-sectional view of cylinder 10 showing the cylinder wall 20 and the biodegradable matrix 30 therein.

In a preferred embodiment of the present invention, the device is preferably between 0.1 cm and 3.0 cm in length and preferably between 0.1 cm and 3.0 cm in diameter. It is to be understood that while a cylinder is the preferred shape of the device that is contemplated as the present invention, the device can be any other shape including, but not limited to, a disc, or a ring.

The long-term delivery device can also have a diameter which ranges in size from 20 microns to 3,000 microns and length which can range in size from 20 microns to 3,000 microns. One micron is defined as 1×10⁻⁶ meters. In a more preferred embodiment of the present invention, the device is between 20 to 200 microns in length and between 20 to 200 microns in diameter.

It is to be understood that while a cylinder is the described shape of the device, the device can be any other shape including, but not limited to, a disc, a rectangle or a ring. An embodiment of a disc would have the dimensions of 120 microns in diameter and 120 microns in thickness. An embodiment of a rectangle would be 1.2 mm long, 1.0 mm wide and 1.0 mm thick. An embodiment of a ring would have the dimensions of 1.2 mm outer diameter and 200 microns inner diameter.

The biodegradable matrix that is placed inside the cylinder can be a matrix chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The therapeutic agents can be any compound that is biologically active and requires long term administration to a tissue or organ for maximum efficacy. Therapeutic agents that can be used in accordance with the present invention include, but are not limited to, extended release antibiotics via the urinary bladder implant that are activated by inflammatory PH and inflammatory mediators. Representative examples of drugs for the treatment of infections include mitomycin, gentamicin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

The kinetics of the therapeutic agent release are optimally at zero order kinetics or near zero order kinetics. The rate of release of the therapeutic agent will vary depending upon the target tissue or organ and on the therapeutic agent or agents being delivered. The rate of release of the therapeutic agent can be controlled by the choice of active ingredients with different solubilities and biodegradable matrix, how the therapeutic agent is physically trapped or chemically immobilized in the biodegradable matrix, by varying the area of the biodegradable matrix exposed to the tissue by adjusting the area of the opening in the container, and/or adjusting the permeability of the walls and/or opening to the therapeutic agent.

As will be appreciated, the extended release extended release therapeutics for urinary tract infection can be delivered using other urinary bladder implants known in the art, for example, in certain embodiments, the implantable device is selected from stents, grafts, stent-grafts, catheters, shunts, closure devices, valves, and particles. Exemplary devices are found, for example, in U.S. Pat. Nos. 4,016,251, 4,016,251; 8,343,516; U.S. Pat. Pub. No. 2004/0260272; and U.S. Pat. Pub. No. 2012/0190636, the entirety of each of which is incorporated by reference hereby.

In some embodiments, the device may be deployed in a minimally invasive procedure, such as by implanting the device through the urethra into the ureter so that the drug delivery portion becomes implanted in the bladder.

1. An extended-release therapeutic for the treatment of urinary tract infection in-vivo comprising a biodegradable matrix and an antibiotic activated by inflammatory PH and inflammatory mediators.

2. The extended-release therapeutic of claim 1 wherein the therapeutic comprises a therapeutic agent selected from the group consisting essentially of: mitomycin, gentamicin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

3. A urinary bladder implant comprising a housing for the delivery of an extended-release therapeutic for the treatment of urinary tract infection in-vivo comprising a biodegradable matrix and an antibiotic activated by inflammatory PH and inflammatory mediators.

4. The urinary bladder implant of claim 1 wherein the therapeutic comprises a therapeutic agent selected from the group consisting essentially of: mitomycin, gentamicin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

5. The urinary bladder implant of claim 1 wherein the implantable device is selected from stents, grafts, stent-grafts, catheters, shunts, closure devices, valves, and particles.
